(19) United States
(12) Patent Application Publication
(10) Pub. No.: US 2018/0093982 A1
(43) Pub. Date: Apr. 5, 2018

(54) METHODS AND USES OF COMPOUNDS FOR TREATING DISEASE

(71) Applicants: Prakash NARAYAN, East Northport, NY (US); Brian HUANG, Commack, NY (US); Prani PAKA, Elmont, NY (US); Latha PAKA, Elmont, NY (US); Itzhak D. GOLDBERG, Englewood, NJ (US)

(72) Inventors: Prakash NARAYAN, East Northport, NY (US); Brian HUANG, Commack, NY (US); Prani PAKA, Elmont, NY (US); Latha PAKA, Elmont, NY (US); Itzhak D. GOLDBERG, Englewood, NJ (US)

(73) Assignee: ANGION BIOMEDICA CORP., Uniondale, NY (US)

(21) Appl. No.: 15/488,488

(22) Filed: Apr. 16, 2017

Related U.S. Application Data
(63) Continuation of application No. 14/493,448, filed on Sep. 23, 2014, now abandoned.
(60) Provisional application No. 61/881,374, filed on Sep. 23, 2013.

(51) Int. Cl. C07D 471/04

(52) U.S. Cl. CPC C07D 471/04

(57) ABSTRACT

The present invention provides methods for treating polycystic kidney disease by administering a compound or pharmaceutical composition thereof having the general structural formula (I)

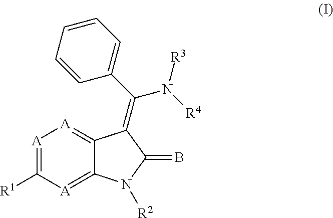

and pharmaceutically acceptable derivatives thereof, as described generally and in classes and subclasses herein.

### CROSS-REFERENCE TO RELATED APPLICATION

This application is a continuation of U.S. patent application Ser. No. 14/493,448, filed Sep. 23, 2014, which claims priority under 35 U.S.C. 119(e) to U.S. patent application Ser. No. 61/881,374, filed Sep. 23, 2013, which is incorporated herein by reference in its entirety.

### GOVERNMENT SUPPORT

This invention was made with U.S. government support under Grant No. DK085841 awarded by the National Institutes of Health. The U.S. government has certain rights in the invention.

### BACKGROUND OF THE INVENTION

Polycystic kidney disease (PKD) is characterized by the progressive formation and enlargement of renal cysts. A number of studies have implicated growth factor signaling (including PDGF and VEGF) in renal cyst formation and expansion. New treatments for PKD are needed.

### SUMMARY OF THE INVENTION

In one embodiment, methods are provided for treating polycystic kidney disease comprising administering to a subject in need thereof a compound or pharmaceutical composition thereof having the structure shown in Formula (I) below:

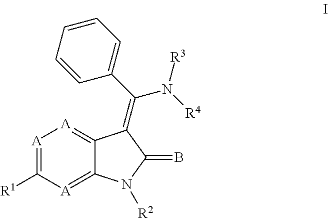

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group;
A is N or CH, wherein one A is nitrogen; and
B is O or S.

In another embodiment, methods are provided for treating polycystic kidney disease comprising administering to a subject in need thereof a compound or pharmaceutical composition thereof having the structure shown in Formula (II) below:

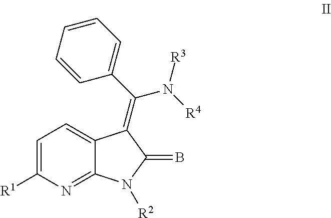

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

In still another embodiment, methods are provided for treating polycystic kidney disease comprising administering to a subject in need thereof a compound or pharmaceutical composition thereof having the structure shown in Formula (III) below:

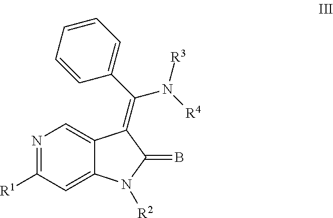

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

In yet another embodiment, methods are provided for treating polycystic kidney disease comprising administering to a subject in need thereof a compound or pharmaceutical composition thereof having the structure shown in Formula (IV) below:

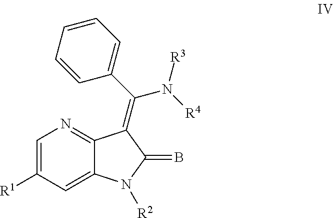

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

In another aspect, the present invention is directed to a method of prevention, treatment or lessening of the severity of polycystic kidney disease, comprising administering to a subject in need thereof an effective amount of a compound of Formula (I-IV) or a pharmaceutical composition thereof.

In another aspect, the present invention is directed to a method of prevention, treatment or lessening of the severity of polycystic disease in other organs such as but not limited to the liver and pancreas, comprising administering to a subject in need thereof an effective amount of a compound of Formula (I-IV) or a pharmaceutical composition thereof.

### DEFINITIONS

It is understood that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbanked, carbocyclic and heterocyclic, aromatic and non-aromatic, carbon and heteroatom substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment and prevention, for example of disorders, as described generally above. Examples of substituents include, but are not limited to aliphatic; heteroaliphatic; alicyclic; heterocyclic; aromatic, heteroaromatic; aryl; heteroaryl; alkylaryl; aralkyl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or -GR^{G1} wherein G is —O—, —S—, —NR^{G2}—, —C(═O)—, S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety. Additional examples of generally applicable substituents are illustrated by the specific embodiments shown in the Examples that are described herein.

The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched) or branched aliphatic hydrocarbons as defined by IUPAC, which are optionally substituted with one or more functional groups. As defined herein, “aliphatic” is intended to include optionally substituted alkyl, alkenyl and alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having about 1-6 carbon atoms. In some instances aliphatic can include alicyclic or cycloalkyl, including unsaturations therein.

In certain embodiments, the alkyl, alkenyl and alkynyl groups employed in the invention contain 1-20; 2-20; 3-20; 4-20; 5-20; 6-20; 7-20 or 8-20 aliphatic carbon atoms. In certain other embodiments, the alkyl, alkenyl, and alkynyl groups employed in the invention contain 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl, alkenyl, and alkynyl groups employed in the invention contain 1-6; 2-6; 3-6; 4-6 or 5-6 aliphatic carbon atoms. In yet other embodiments, the alkyl, alkenyl, and alkynyl groups employed in the invention contain 1-4; 2-4 or 3-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargy1), 1-propynyl and the like.

The term “alicyclic”, as used herein, refers to compounds that combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norbornyl moieties and the like, which again, may bear one or more substituents.

The term “cycloalkyl”, as used herein, refers to cyclic alkyl groups, specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, heteroaliphatic or heterocyclic moieties, may optionally be substituted. An analogous convention applies to other generic terms such as “cycloalkenyl”, “cycloalkynyl” and the like. Additional examples of generally applicable substituents are illustrated by the specific embodiments shown in the Examples that are described herein.

The term “heteroaliphatic”, as used herein, refers to aliphatic moieties in which one or more carbon atoms in the main chain have been replaced with a heteroatom. Thus, a heteroaliphatic group refers to an aliphatic chain which contains one or more oxygen, sulfur, nitrogen, phosphorus or silicon atoms in place of carbon atoms in the aliphatic main chain. Heteroaliphatic moieties may be branched or linear unbranched. In certain embodiments, heteroaliphatic moieties are substituted by independent replacement of one or more of the hydrogen atoms thereon with one or more moieties including, but not limited to aliphatic; heteroaliphatic; alicyclic; heterocyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety. Additional examples of generally applicable substituents are illustrated by the specific embodiments shown in the Examples that are described herein.

The term “heteroalicyclic”, “heterocycloalkyl” or “heterocyclic”, as used herein, refers to compounds which combine the properties of heteroaliphatic and cyclic compounds and include but are not limited to saturated and unsaturated mono- or polycyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocyclic” refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 2 double bonds and each 6-membered ring has 0 to 2 double bonds, (ii) the nitrogen and sulfur heteroatoms may optionally be oxidized, (iii) the nitrogen heteroatom may optionally be quaternized, and (iv) any of the above heterocyclic rings may be fused to an aryl or heteroaryl ring. Representative heterocycles include, but are not limited to, pyrrolidinyl, pyrazolinyl, pyrazolidinyl imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one or more hydrogen atoms thereon with aliphatic; heteroaliphatic; alicyclic; heterocyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or -GR^{G1} wherein G is —O—, —S—, —NR^{G2}—, —C(═O)—, —S(═O)—, —SO₂—, —C(═O)O—, —C(═O)NR^{G2}—, —OC(═O)—, NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”. Examples of aromatic moieties include, but are not limited to, phenyl, indanyl, indenyl, naphthyl, phenanthryl and anthracyl.

In general, the term “heteroaromatic moiety”, as used herein, refers to stable substituted or unsubstituted unsaturated mono-heterocyclic or polyheterocyclic moieties having preferably 3-14 carbon atoms, comprising at least one ring having p-orbitals perpendicular to the plane of the ring at each ring atom, and satisfying the Huckel rule where the number of pi electrons in the ring is (4n+2) wherein n is an integer. Examples of heteroaromatic moieties include, but are not limited to, pyridyl, quinolinyl, dihydroquinolinyl, isoquinolinyl, quinazolinyl, dihydroquinazolyl, and tetrahydroquinazolyl.

It will also be appreciated that aromatic and heteroaromatic moieties, as defined herein, may be attached via an aliphatic (e.g., alkyl) or heteroaliphatic (e.g., heteroalkyl) moiety and thus also include moieties such as -(aliphatic)aromatic, -(heteroaliphatic)aromatic, -(aliphatic)heteroaromatic, -(heteroaliphatic)heteroaromatic, -(alkyl)aromatic, -(heteroalkyl)aromatic, -(alkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. In some instances corresponding moieties may be referred to synonymously as aralkyl, heteroaralkyl and the like. Substituents include, but are not limited to, any of the previously mentioned substituents, i.e., the substituents recited for aliphatic moieties, or for other moieties as disclosed herein, resulting in the formation of a stable compound.

In general, the term “aryl” refers to aromatic moieties, as described above, excluding those attached via an aliphatic (e.g., alkyl) or heteroaliphatic (e.g., heteroalkyl) moiety. In certain embodiments of the present invention, “aryl” refers to a mono- or bicyclic carbocyclic ring system having one or two rings satisfying the Huckel rule for aromaticity, including, but not limited to, phenyl, naphthyl, tetrahydronaphthyl, indanyl, indenyl and the like.

Similarly, the term “heteroaryl” refers to heteroaromatic moieties, as described above, excluding those attached via an aliphatic (e.g., alkyl) or heteroaliphatic (e.g., heteroalkyl) moiety. In certain embodiments of the present invention, the term “heteroaryl”, as used herein, refers to a cyclic unsaturated radical having from about five to about ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

As defined herein, “aryl” and “heteroaryl” groups (including bicyclic aryl groups) can be unsubstituted or substituted, wherein substitution includes replacement of one or more of the hydrogen atoms thereon independently with any of the previously mentioned substitutents, i.e., the substituents recited for aliphatic moieties, or for other moieties as disclosed herein, resulting in the formation of a stable compound. For example, aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; heteroaliphatic; alicyclic; heterocyclic; aromatic, heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —NO₂; —CN; —CF₃; —CH₂CF₃; —CHCl₂; —CH₂OH; —CH₂CH₂OH; —CH₂NH₂; —CH₂SO₂CH₃; or -GR^{G1} wherein G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G2}—, —C(═S)—, —C(═S)S—, —SC(═S)—, —SC(═S)S—, —C(═NR^{G2})—, —C(═NR^{G2})O—, —C(═NR^{G2})NR^{G3}—, —OC(═NR^{G2})—, —NR^{G2}C(═NR^{G3})—, —NR^{G2}SO₂—, —NR^{G2}SO₂NR^{G3}—, or —SO₂NR^{G2}—, wherein each occurrence of R^{G1}, R^{G2} and R^{G3} independently includes, but is not limited to, hydrogen, halogen, or an optionally substituted aliphatic, heteroaliphatic, alicyclic, heterocyclic, aromatic, heteroaromatic, aryl, heteroaryl, alkylaryl, or alkylheteroaryl moiety. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20; 2-20; 3-20; 4-20; 5-20; 6-20; 7-20 or 8-20 aliphatic carbon atoms. In certain other embodiments, the alkyl group contains 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl group contains 1-6; 2-6; 3-6; 4-6 or 5-6 aliphatic carbon atoms. In yet other embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like.

The term “alkylamino” refers to a group having the structure —NHR′ wherein R′ is aliphatic or alicyclic, as defined herein. The term “aminoalkyl” refers to a group having the structure NH₂R′—, wherein R′ is aliphatic or alicyclic, as defined herein. In certain embodiments, the aliphatic or alicyclic group contains 1-20 aliphatic carbon atoms. In certain other embodiments, the aliphatic or alicyclic group contains 1-10 aliphatic carbon atoms. In still other embodiments, the aliphatic or alicyclic group contains 1-6 aliphatic carbon atoms. In yet other embodiments, the aliphatic or alicyclic group contains 1-4 aliphatic carbon atoms. In yet other embodiments, R′ is an alkyl, alkenyl, or alkynyl group containing 1-8 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SRₓ; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “C₂₋₆alkenylene”, as used herein, refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocycloalkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift.

By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-alkyl and N-aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

As used herein, the term “isolated” when applied to the compounds of the present invention, refers to such compounds that are (i) separated from at least some components with which they are associated in nature or when they are made and/or (ii) produced, prepared or manufactured by the hand of man.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof; or purified versions thereof. For example, the term “biological sample” refers to any solid or fluid sample obtained from, excreted by or secreted by any living organism, including single-celled microorganisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, seminal fluid, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### BRIEF DESCRIPTION OF THE FIGURES

FIG. 1 shows that in a PKD model, inventive compound administered for 4 weeks after disease onset prevents the increase in kidney size consequent to PKD;

FIG. 2 shows the results in FIG. 2 in graphic format, as well as the effect of compound on the ratio of kidney to body mass and cystic index;

FIG. 3 shows that inventive compound demonstrated a significant prevention of proteinuria, NGAL and Kim-1 after 4 weeks of administration;

FIG. 4 shows that albuminuria and kidney hydroxyproline are significantly benefited by 4 weeks of treatment;

FIG. 5 shows that after 7 weeks of treatment, compound administration improves cystic index;

FIG. 6 shows that BUN and serum creatinine are improved after 7 weeks of compound administration;

FIG. 7 shows that hydroxyproline content of the kidney is reduced after 7 weeks of treatment;

FIG. 8 shows that proteinuria, NGAL, albuminuria and Kim-1 are reduced by 7 weeks of compound administration; and

FIG. 9 shows that formation of liver cysts in this model are reduced, as well as liver function is improved and hydroxyproline in the liver is reduced, following 7 weeks of treatment.

### DETAILED DESCRIPTION OF CERTAIN PREFERRED EMBODIMENTS OF THE INVENTION

Polycystic kidney disease (PKD) is characterized by the progressive formation and enlargement of renal cysts. A number of studies have implicated growth factor signaling (including PDGF and VEGF) in renal cyst formation and expansion. Compounds described herein have been found useful for the prevention, treatment or lessening the severity of PKD. Polycystic disease also can affect other organs such as the liver, pancreas, heart and brain, and compounds described herein are also useful for preventing, treating or lessening of the severity of polycystic disease in other organs such as but not limited to the liver and pancreas.

Thus, in one embodiment, methods are provided for treating polycystic kidney disease comprising administering to a subject in need thereof a compound or pharmaceutical composition thereof having the structure shown in Formula (I) below:

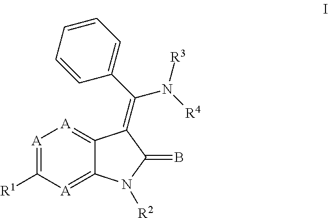

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group;
A is N or CH, wherein one A is nitrogen; and
B is O or S.

In certain embodiments, R² is hydrogen.

In certain embodiments, R² is a carbamate prodrug moiety.

In certain embodiments, R² is an amide prodrug moiety.

In certain embodiments, R³ is hydrogen.

In certain embodiments, R⁴ is aryl, such as phenyl group.

In certain embodiments, R⁵ is lower alkyl, such as methyl group.

In certain embodiments, R⁶ is alkyl, such as methyl.

In certain embodiments, R⁷ is alkylheterocycloalkyl, such as methyl piperazinylmethyl.

In certain embodiments, R⁸ is lower alkyl, such as methyl.

In certain embodiments, R⁹ is hydrogen.

In certain embodiments the A at position 4 is N, and the other occurrences of A are C. In certain other embodiments, the A at position 5 is N, and the other occurrences of A are C. In another embodiment, the A at position 7 is N, and the other occurrences of A are C.

Non-limiting examples of compounds of formula (I) useful for treating PKD include: (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate; (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.

In another embodiment, methods are provided for treating polycystic kidney disease comprising administering to a subject in need thereof a compound or pharmaceutical composition thereof having the structure shown in Formula (II) below:

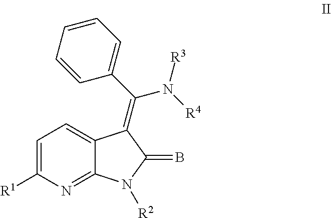

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

In certain embodiments, R² is hydrogen.

In certain embodiments, R² is a carbamate prodrug moiety.

In certain embodiments, R² is an amide prodrug moiety.

In certain embodiments, R³ is hydrogen.

In certain embodiments, R⁴ is aryl, such as phenyl group.

In certain embodiments, R⁵ is lower alkyl, such as methyl group.

In certain embodiments, R⁶ is alkyl, such as methyl.

In certain embodiments, R⁷ is alkylheterocycloalkyl, such as methyl piperazinylmethyl.

In certain embodiments, R⁸ is lower alkyl, such as methyl.

In certain embodiments, R⁹ is hydrogen.

Non-limiting examples of compounds of formula (II) useful for treating PKD include: (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate.

In still another embodiment, methods are provided for treating polycystic kidney disease comprising administering to a subject in need thereof a compound or pharmaceutical composition thereof having the structure shown in Formula (III) below:

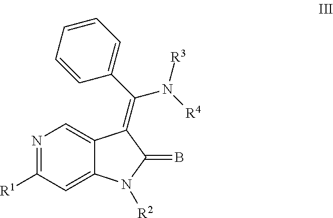

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

In certain embodiments, R² is hydrogen.

In certain embodiments, R² is a carbamate prodrug moiety.

In certain embodiments, R² is an amide prodrug moiety.

In certain embodiments, R³ is hydrogen.

In certain embodiments, R⁴ is aryl, such as phenyl group.

In certain embodiments, R⁵ is lower alkyl, such as methyl group.

In certain embodiments, R⁶ is alkyl, such as methyl.

In certain embodiments, R⁷ is alkylheterocycloalkyl, such as methyl piperazinylmethyl.

In certain embodiments, R⁸ is lower alkyl, such as methyl.

In certain embodiments, R⁹ is hydrogen.

Non-limiting examples of compounds of formula (III) useful for treating PKD include: (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate.

In yet another embodiment, methods are provided for treating polycystic kidney disease comprising administering to a subject in need thereof a compound or pharmaceutical composition thereof having the structure shown in Formula (IV) below:

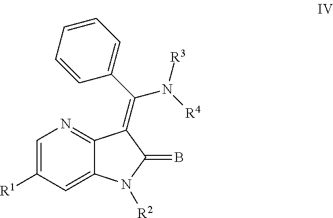

or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

In certain embodiments, R² is hydrogen.

In certain embodiments, R² is a carbamate prodrug moiety.

In certain embodiments, R² is an amide prodrug moiety.

In certain embodiments, R³ is hydrogen.

In certain embodiments, R⁴ is aryl, such as phenyl group.

In certain embodiments, R⁵ is lower alkyl, such as methyl group.

In certain embodiments, R⁶ is alkyl, such as methyl.

In certain embodiments, R⁷ is alkylheterocycloalkyl, such as methyl piperazinylmethyl.

In certain embodiments, R⁸ is lower alkyl, such as methyl.

In certain embodiments, R⁹ is hydrogen.

Non-limiting examples of compounds of formula (IV) useful for treating PKD include: (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.

The above Formulas (I), (II), (III) and (IV) are shown without a definitive stereochemistry at certain positions. The present invention includes all stereoisomers of Formulas (I), (II), (III) and (IV) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be mixture of stereoisomers.

It will be appreciated that for each of the classes and subclasses described above and herein, any one or more occurrences of aliphatic and/or heteroaliphatic may independently be substituted or unsubstituted, linear or branched, saturated or unsaturated; any one or more occurrences of alicyclic and/or heteroalicyclic may independently be substituted or unsubstituted, saturated or unsaturated; and any one or more occurrences of aryl and/or heteroaryl may independently be substituted or unsubstituted.

Some of the foregoing compounds can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization of compound of Formula (I), (II), (III) and (IV) under different conditions and may exist as one or a combination of polymorphs of compound of general formulas (I), (II), (III) and (IV) forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric and geometrical isomeric forms, their stereoisomers, their positional isomer, their polymorphs, their pharmaceutically acceptable salts their pharmaceutically acceptable solvates and pharmaceutically acceptable compositions containing them. Tautomeric forms of compounds of the present invention include, pyrazoles, pyridones and enols, etc., and geometrical isomers include E/Z isomers of compounds having double bonds and cis-trans isomers of monocyclic or fused ring systems, etc.

### 2) Pharmaceutical Compositions

As discussed above this invention provides novel compounds that have biological properties useful for the treatment of polycystic kidney disease, as well as polycystic disease of other organs.

Accordingly, in another aspect of the present invention, pharmaceutical compositions are provided, which comprise any one or more of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved agent to treat the same or related indication, or it may be any one of a number of agents undergoing approval in the Food and Drug Administration that ultimately obtain approval for the treatment of any disorder related to PKD. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemi sulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, or N-demethylation of a compound of the invention where R¹ is methyl. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. By way of other examples, carbamate and amide prodrugs of compounds of formulae (I)-(IV) are embodied herein, such as those discussed in Rautio et al., 2008, Nature Rev Drug Discov 7:255-70; Jordan et al., 2003, Bioorg Med Chem 10:2625-33 and Hay et al., 2003, J Med Chem 46:5533-45, by way of non-limiting examples.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In other embodiments solid dosage forms are provided. In certain embodiments, such solid dosage forms provide a higher than about a 20% oral bioavailability. As will be shown in the examples below, compounds of the invention can be co-precipitated with one or more agents such as mannitol, a combination of mannitol and lactobionic acid, a combination of mannitol and gluconic acid, a combination of mannitol and methanesulfonic acid, a combination of microcrystalline cellulose and oleic acid or a combination of pregelatinized starch and oleic acid. The foregoing examples of one or more agents to aid in preparing formulations of inventive compound are merely illustrative and non-limiting. Non-limiting examples of inventive compounds in such solid dosage forms include

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfate, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Formulations for intraocular administration are also included. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anti-inflammatory agent), or they may achieve different effects (e.g., control of any adverse effects). In non-limiting examples, one or more compounds of the invention may be formulated with at least one cytokine, growth factor or other biological, such as an interferon, e.g., alpha interferon, or with at least another small molecule compound. Non-limiting examples of pharmaceutical agents that may be combined therapeutically with compounds of the invention include: antivirals and antifibrotics such as interferon alpha, combination of interferon alpha and ribavirin, Lamivudine, Adefovir dipivoxil and interferon gamma; anticoagulants such as heparin and warfarin; antiplatelets e.g., aspirin, ticlopidine and clopidogrel; other growth factors involved in regeneration, e.g., VEGF and FGF and mimetics of these growth factors ; antiapoptotic agents; and motility and morphogenic agents.

In certain embodiments, the pharmaceutical compositions of the present invention further comprise one or more additional therapeutically active ingredients (e.g., anti-inflammatory and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs.

### 3) Pharmaceutical Uses and Methods of Treatment

In certain embodiments, the method of treating PKD involves the administration of a therapeutically effective amount of the compound or a pharmaceutically acceptable derivative thereof to a subject (including, but not limited to a human or animal) in need of it. Subjects for which the benefits of the compounds of the invention are intended for administration include, in addition to humans, livestock, domesticated, zoo and companion animals.

It will be appreciated that the compounds and compositions, according to the method of the present invention, may be administered using any amount and any route of administration effective for the treatment of PKD. Thus, the expression “effective amount” as used herein, refers to a sufficient amount of agent to exhibit a therapeutic effect. The exact amount required will vary from subject to subject, depending on the species, age, and general condition of the subject, the severity of the infection, the particular therapeutic agent, its mode and/or route of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, subcutaneously, intradermally, intra-ocularly, topically (as by powders, ointments, or drops), buccally, as an oral or nasal spray, or the like, depending on the severity of the disease or disorder being treated. In certain embodiments, the compounds of the invention may be administered at dosage levels of about 0.001 mg/kg to about 50 mg/kg, preferably from about 0. 1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

### 1) General Description of Synthetic Methods:

The practitioner has a well-established literature of small molecule chemistry to draw upon, in combination with the information contained herein, for guidance on synthetic strategies, protecting groups, and other materials and methods useful for the synthesis of the compounds of this invention.

The various references cited herein provide helpful background information on preparing compounds similar to the inventive compounds described herein or relevant intermediates, as well as information on formulation, uses, and administration of such compounds which may be of interest.

Moreover, the practitioner is directed to the specific guidance and examples provided in this document relating to various exemplary compounds and intermediates thereof.

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

According to the present invention, any available techniques can be used to make or prepare the inventive compounds or compositions including them. For example, a variety of solution phase synthetic methods such as those discussed in detail below may be used. Alternatively or additionally, the inventive compounds may be prepared using any of a variety combinatorial techniques, parallel synthesis and/or solid phase synthetic methods known in the art.

It will be appreciated as described below, that a variety of inventive compounds can be synthesized according to the methods described herein. The starting materials and reagents used in preparing these compounds are either available from commercial suppliers such as Aldrich Chemical Company (Milwaukee, Wis.), Bachem (Torrance, Calif.), Sigma (St. Louis, Mo.), or are prepared by methods well known to a person of ordinary skill in the art following procedures described in such references as Fieser and Fieser 1991, “Reagents for Organic Synthesis”, vols 1-17, John Wiley and Sons, New York, N.Y., 1991; Rodd 1989 “Chemistry of Carbon Compounds”, vols. 1-5 and supps, Elsevier Science Publishers, 1989; “Organic Reactions”, vols 1-40, John Wiley and Sons, New York, N.Y., 1991; March 2001, “Advanced Organic Chemistry”, 5th ed. John Wiley and Sons, New York, N.Y.; and Larock 1990, “Comprehensive Organic Transformations: A Guide to Functional Group Preparations”, 2^{nd} ed. VCH Publishers. These schemes are merely illustrative of some methods by which the compounds of this invention can be synthesized, and various modifications to these schemes can be made and will be suggested to a person of ordinary skill in the art having regard to this disclosure. Further guidance for the preparation of the compounds useful for the purposes described herein for can be found, for example, in PCT/US2013/023324, published as WO2013/112959.

The starting materials, intermediates, and compounds of this invention may be isolated and purified using conventional techniques, including filtration, distillation, crystallization, chromatography, and the like. They may be characterized using conventional methods, including physical constants and spectral data.

### General Reaction Procedures:

Unless mentioned specifically, reaction mixtures are stirred using a magnetically driven stirrer bar. An inert atmosphere refers to either dry argon or dry nitrogen. Reactions are monitored either by thin layer chromatography, by proton nuclear magnetic resonance (NMR) or by high-pressure liquid chromatography (HPLC), of a suitably worked up sample of the reaction mixture.

### General Work Up Procedures:

Unless mentioned specifically, reaction mixtures are cooled to room temperature or below then quenched, when necessary, with either water or a saturated aqueous solution of ammonium chloride. Desired products are extracted by partitioning between water and a suitable water-immiscible solvent (e.g. ethyl acetate, dichloromethane, diethyl ether). The desired product containing extracts are washed appropriately with water followed by a saturated solution of brine. On occasions where the product containing extract is deemed to contain residual oxidants, the extract is washed with a 10% solution of sodium sulphite in saturated aqueous sodium bicarbonate solution, prior to the aforementioned washing procedure. On occasions where the product containing extract is deemed to contain residual acids, the extract is washed with saturated aqueous sodium bicarbonate solution, prior to the aforementioned washing procedure (except in those cases where the desired product itself had acidic character). On occasions where the product containing extract is deemed to contain residual bases, the extract is washed with 10% aqueous citric acid solution, prior to the aforementioned washing procedure (except in those cases where the desired product itself had basic character). Post washing, the desired product containing extracts are dried over anhydrous magnesium sulphate, and then filtered. The crude products are then isolated by removal of solvent(s) by rotary evaporation under reduced pressure, at an appropriate temperature (generally less than 45° C.).

### General Purification Procedures:

Unless mentioned specifically, chromatographic purification refers to flash column chromatography on silica, using a single solvent or mixed solvent as eluent. Suitably purified desired product containing elutes are combined and concentrated under reduced pressure at an appropriate temperature (generally less than 45° C.) to constant mass.

### 1) Synthesis of Exemplary Compounds:

### Example 1

### (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate

Step-1: To a solution of methyl 2-oxo-2,3-dihydro-1H-pyrrolo[2,3-b]pyridine-6-carboxylate (1 g, 5.20 mmol) in AC₂O (10 mL) was added triethyl orthobenzoate (3.40 g, 15.59 mmol) at RT and the mixture was heated to reflux for 3 h. The reaction mixture was evaporated and the resultant residue was purified by silica gel column chromatography using 5% CH₃OH in dichloromethane as eluent to afford (E)-methyl 1-acetyl-3-(ethoxy(phenyl)methylene)-2-oxo-2,3-dihydro-1H-pyrrolo[2,3-b]pyridine-6-carboxylate as an orange solid. ¹H NMR (CDCl₃, 500 MHz): δ 8.25 (d, J=12.1 Hz, 1 H), 8.04 (d, J=12.1 Hz, 1H), 7.53-7.60 (m, 3H), 7.38-7.45 (m, 2H), 4.40 (q, J=7.1 Hz, 2H), 3.99 (s, 3H), 2.63 (s, 3H), 1.42 (t, J=7.1 Hz, 3H).

Step-2: To a solution of (E)-methyl 1-acetyl-3-(ethoxy(phenyl)methylene)-2-oxo-2,3-dihydro-1H-pyrrolo[2,3-b]pyridine-6-carboxylate (2.6 g, 7.10 mmol) in DMF (5 mL) was added N-(4-aminophenyl)-N-methyl-2-(4-methylpiperazin-1-yl)acetamide (1.94 g, 7.43 mmol) at RT and the reaction mixture was heated to 110° C. and stirred for 1 h. The reaction mixture was allowed to cool to RT, treated with piperidine (3 mL) and stirred for 30 min. The reaction mixture was evaporated and the resultant residue was purified by silica gel column chromatography using 5% CH₃OH in dichloromethane as eluent to afford (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate as yellow solid. MS (ES+): m/z 541.1 (MH⁺).

### Example 2

### (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate

Step-1: To a solution of methyl 2-oxo-2,3-dihydro-1H-pyrrolo[3,2-c]pyridine-6-carboxylate (10 mg, 0.052 mmol) in toluene (5 mL) was added Ac₂O (15 mg, 0.156 mmol) and triethyl orthobenzoate (35 mg, 0.156 mmol) at room temperature. The reaction mixture was heated stirred at 85° C. for 3 h. The solvent was evaporated under reduced pressure and the resulting residue was purified by silica gel column chromatography using 5% acetone in dichloromethane to afford (E)-methyl 1-acetyl-3-(ethoxy(phenyl)methylene)-2-oxo-2,3-dihydro-1H-pyrrolo[3,2-c]pyridine-6-carboxylate as yellow solid. ¹H NMR (CDCl₃, 400 MHz) δ 9.15 (s, 1H), 8.67 (s, 1H), 7.52-7.59 (m, 5H), 4.06 (dd, J=13.9, 7 Hz), 3.89 (s, 3H), 2.45 (s, 3H), 1.37 (t, J=7 Hz, 3H).

Step-2: To a solution of (E)-methyl 1-acetyl-3-(ethoxy(phenyl)methylene)-2-oxo-2,3-dihydro-1H-pyrrolo[3,2-c]pyridine-6-carboxylate (40 mg, 0.109 mmol) in DMF (0.2 mL) was added N-(4-aminophenyl)-N-methyl-2-(4-methylpiperazin-1-yl)acetamide (28 mg, 0.109 mmol) at room temperature, and the reaction mixture was stirred at 110° C. for 2 h. The reaction mixture was cooled to RT, piperidine (0.2 mL) was added and stirred for 10 minutes at room temperature. The solvent was evaporated and the resulting residue was purified by silica gel column chromatography using 5% CH₃OH in dichloromethane as eluent to afford (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate as yellow solid. MS (ES+): m/z 541.3 (MH⁺).

### Example 3

### (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate

Step-1: To a stirred solution of methyl 2-oxo-2,3-dihydro-1H-pyrrolo[3,2-b]pyridine-6-carboxylate (0.18 g, 0.9366 mmol) in acetic anhydride (4 ml) was added triethyl orthobenzoate (0.630 g, 2.8098 mmol) at RT and the mixture was refluxed for 3 h at 110° C. The reaction mixture was evaporated and the resulting residue was used as such into next step without purification.

Step-2: To a stirred solution of product from step-1 (0.18 g) in DMF (4 ml) was added N-(4-aminophenyl)-N-methyl-2-(4-methylpiperazin-1-yl)acetamide (0.180 g, 0.6861 mmol) at RT and the reaction mixture was heated at 110° C. for 1 h. The reaction mixture was cooled to RT and stirred with triethyl amine (1 ml) for half an hour. The reaction mixture was evaporated and the crude product was purified by column chromatography using 0 to 10% methanol in dichloromethane as eluent to afford (Z)-methyl 1-acetyl-3-((4-(N-methyl-2-(4-methylpiperazin-1-yl) acetamido)phenylamino)(phenyl)methylene)-2-oxo-2,3-dihydro-1H-pyrrolo[3,2-b]pyridine-6-carboxylate as yellow solid. MS (ES+): m/z 583.4 (MH⁺).

Step-3: A mixture of (Z)-methyl 1-acetyl-3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate (6 mg, 0.0103 mmol), K₂CO₃ (1.42 mg, 0.0103 mmol) in MeOH (2 mL) was stirred for 2 h at room temperature. The crude product was concentrated and purified by preparative TLC to afford (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate. MS (ES+): m/z 541.3 (MH)⁺

By following the aforementioned procedure, the following compounds can also be prepared:
(Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate; (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.

The foregoing are merely exemplary of synthetic routes to the compound of the invention. The foregoing compounds, compositions and methods of the invention are illustrated by the following examples, which are merely exemplary of aspects of the invention and are not limiting.

### Example 4

### Compounds of the Invention are Potent Against KDR and PDGFR

Potency of compounds of the invention against the VEGF receptor KDR and the PDGF receptors PDGFRα and PDGFRβ were evaluated in in vitro screens. Compounds of the invention potently inhibit KDR with an IC₅₀ of <10 μM, and PDGFRβ with an IC₅₀ of <10 μM. Kd determination of an exemplary compound against KDR and PDGFRβ showed Kd values of <5 nM and <10 nM respectively. To determine selectivity, an exemplary compound was tested for inhibitory activity against >400 other kinases in the same assay format and found that exemplary compound at 100 nM show <50% inhibition of >95% of the kinases tested.

### Example 5

### Compounds of the Invention Inhibit KDR and PDGFR Signaling

To evaluate the cellular activity of inventive compounds in inhibiting KDR and PDGF receptors, activity on VEGF-induced KDR phosphorylation in human endothelial cells (HUVEC) and PDGF-induced PDGFRβ phosphorylation in human hepatic stellate cells (HSC) was evaluated by Western Analysis. Test compound dose-dependently inhibited the phosphorylation of KDR and PDGFRβ. These observations confirm inventive compounds as inhibitors of the cellular activity of KDR and PDGFRβ.

### Example 6

### Efficacy in Polycystic Kidney Disease

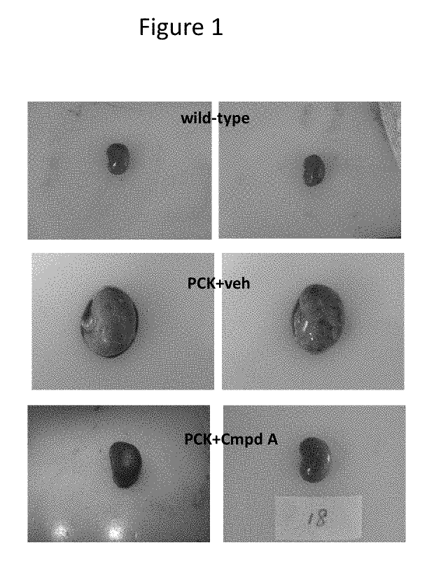

A proof-of-concept study was conducted in the polycystic kidney (PCK) rat (PCK/CrljCrl-pkhd1pck/Crl, Charles River Labs), a highly aggressive model of PKD. Age-matched CD rats were used as controls. Study design was interventional as rats were randomized to vehicle or compound of the invention (compound A; 25 mg/kg, BID, PO) at 6.5 weeks of age following confirmation of frank renal disease. In this experiment, drug effects were studied over 4 weeks of dosing (n=14/group) and are presented herein in FIGS. 1-4.

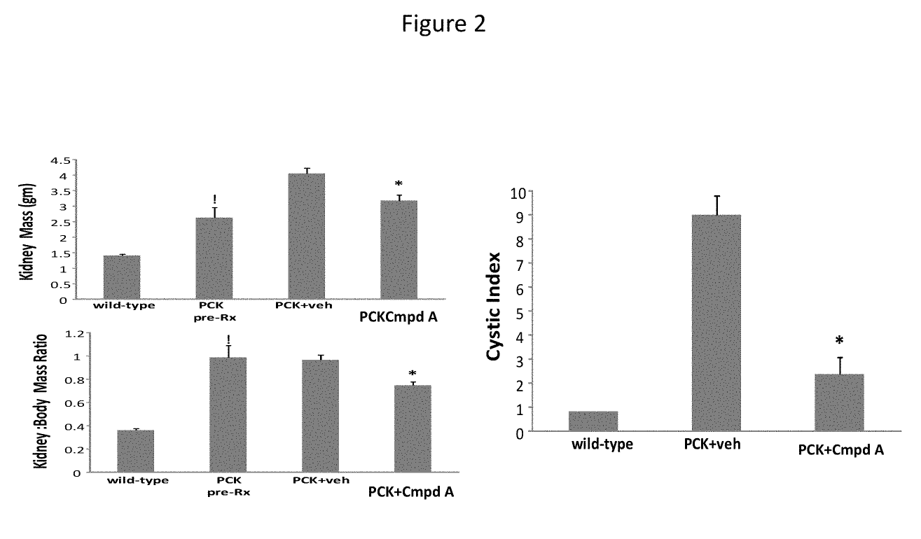

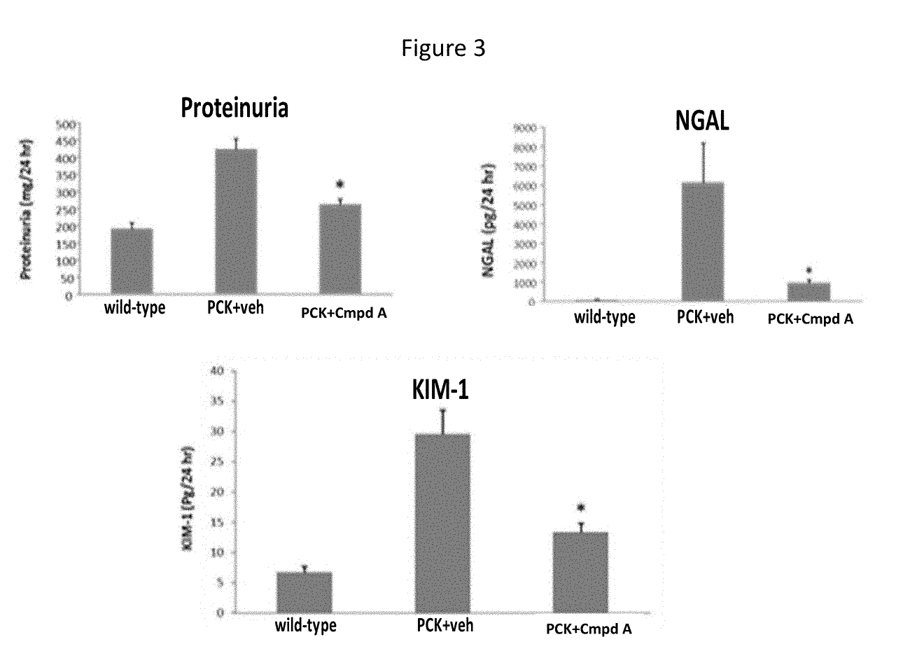

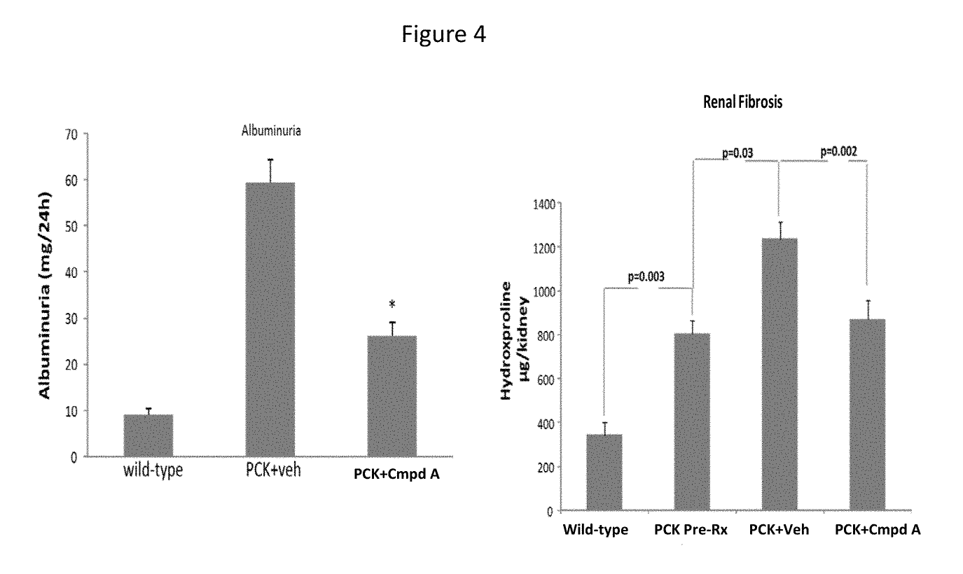

FIG. 1 shows that in the model, inventive compound (Cmpd A) prevents the increase in kidney size consequent to PKD. These results are shown graphically in FIG. 2, top left, as kidney mass, and bottom left, as the ratio of kidney to body mass. In FIG. 2, right, these data are presented as cystic index, the cyst area as a percent of renal area in sagittal kidney section, as measured by an observer blinded to treatment. The positive effect of inventive compound was shown on proteinuria (FIG. 3 top left), NGAL (top right) and Kim-1 (bottom). Effects on proteinuria and renal hydroxyproline are shown in FIG. 4, left and right, respectively.

### Example 7

### Efficacy in Polycystic Kidney Disease

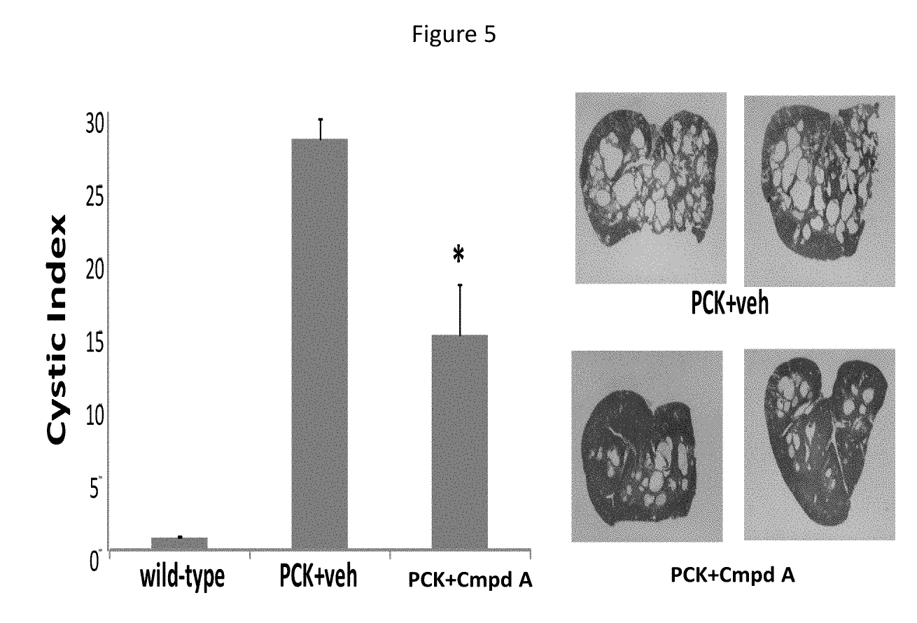

Another study was conducted in the PCK rat (PCK/CrljCrl-pkhd1pck/Crl, Charles River Labs). Similar to that described in Example 6, age-matched CD rats were used as controls, and rats were randomized to vehicle or compound of the invention (compound A; 25 mg/kg, BID, PO) at 6.5 weeks of age following confirmation of frank renal disease. Drug effects were studied over 7 weeks of dosing (n=14/group) and are presented in FIGS. 5-9, using the same analyses as in Example 6.

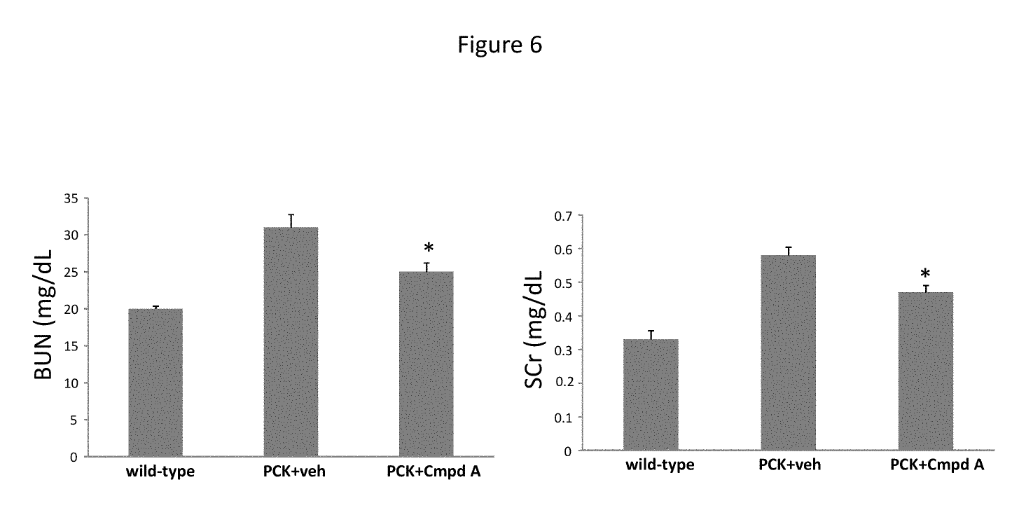

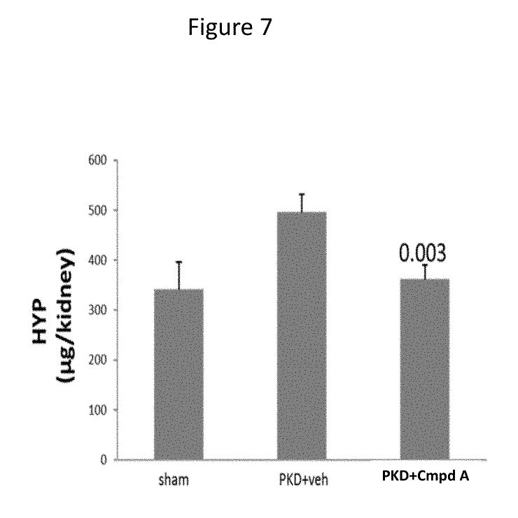

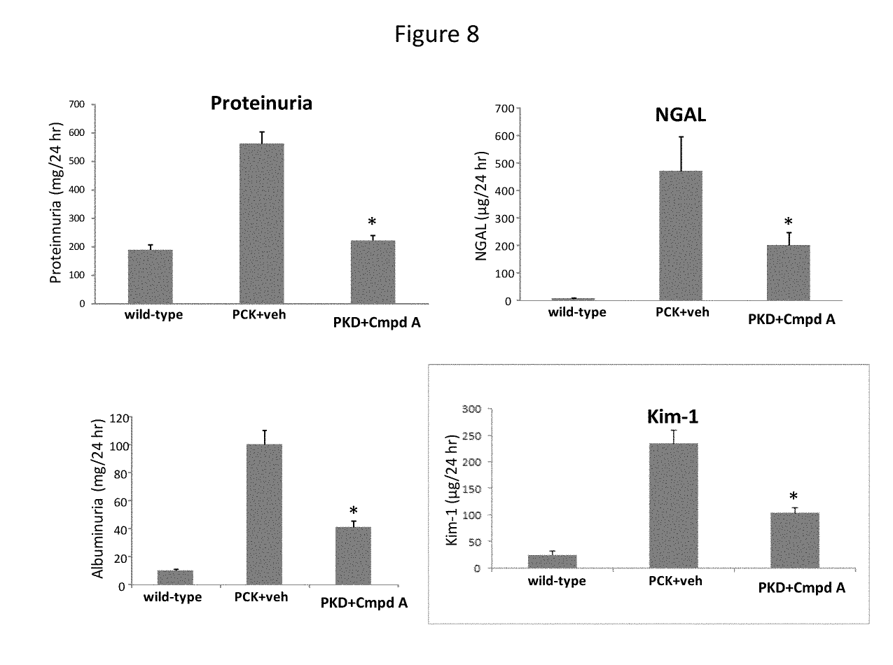

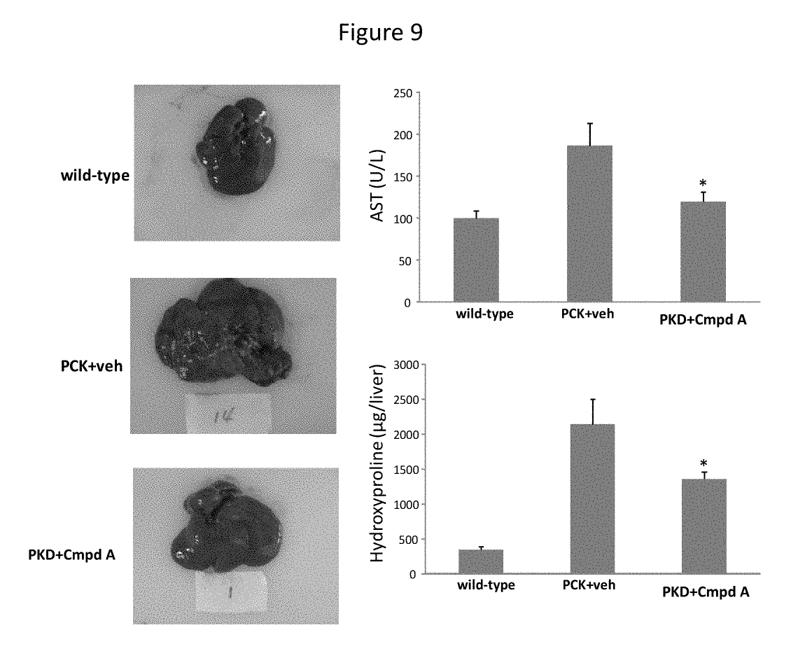

FIG. 5 shows that in the model, inventive compound decreased cystogenesis, using cystic index (left) and in histological sections (right). Measures of renal function, BUN and serum creatinine (Scr) were reduced by compound treatment (FIG. 6, left and right, respectively). Kidney hydroxyproline was significantly reduced (FIG. 7). Proteinuria, NGAL, albuminuria and Kim-1 were all reduced by compound administration (FIG. 8: top left, top right, bottom left ad bottom right, respectively). In addition, this model shows polycystic liver changes as well; FIG. 9 shows that compound treatment reduced abnormal appearance of the liver (left) as well as liver enzyme elevation (AST; top right) and hydroxyproline content (bottom right).

What is claimed is:

1. A method for treating polycystic kidney disease comprising administering to a subject in need thereof a compound or pharmaceutical composition thereof having the structure of Formula (I) below: 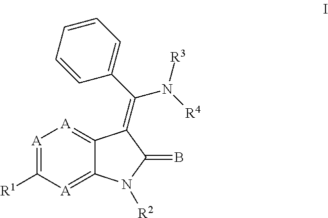
or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group;
A is N or CH, wherein one A is nitrogen; and
B is O or S.

2. The method of claim 1 wherein R² is hydrogen.

3. The method of claim 1 wherein R² is a carbamate prodrug moiety or an amide prodrug moiety.

4. The method of claim 1 wherein R³ is hydrogen.

5. The method of claim 1 wherein R⁴ is phenyl.

6. The method of claim 1 wherein R⁵ is methyl.

7. The method of claim 1 wherein R⁶ is methyl.

8. The method of claim 1 wherein R⁷ is methylpiperazinylmethyl.

9. A method for treating polycystic kidney disease comprising administering to a subject in need thereof a compound or pharmaceutical composition thereof having the structure of Formula Formula (II) below: 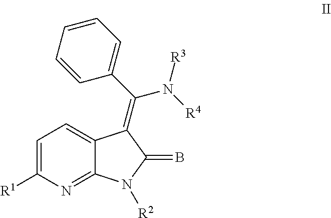
or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

10. The method of claim 9 wherein the compound is selected from (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[2,3-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[2,3-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[2,3-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[2,3-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[2,3-b]pyridine-6-carboxylate.

11. A method for treating polycystic kidney disease comprising administering to a subject in need thereof a compound or pharmaceutical composition thereof having the structure of Formula Formula (III) below: 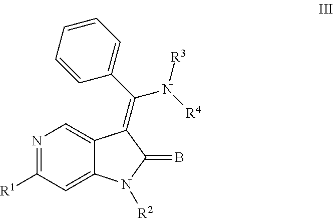
or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is a lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

12. The method of claim 11 wherein the compound is selected from among (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-c]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-c]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-c]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-c]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-c]pyridine-6-carboxylate.

13. A method for treating polycystic kidney disease comprising administering to a subject in need thereof a compound or pharmaceutical composition thereof having the structure of Formula (IV) below: 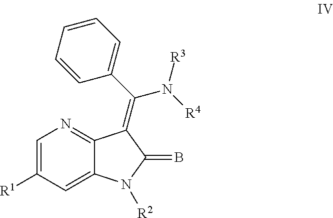
or a pharmaceutically acceptable salt thereof or a prodrug thereof;
wherein R¹ is —COOR⁵;
R² is H or a prodrug moiety, optionally a carbamate or amide;
R³ and R⁴ are independently H, aryl or heteroaryl, which may optionally be independently substituted with one or more lower alkyl, halogen, OR⁶, NO₂, CN, NH₂, NR⁶R⁷, NR⁶COR⁷ or NR⁶SO₂R⁷ moieties;
R⁵ is lower alkyl group;
R⁶ and R⁷ are independently hydrogen or alkyl, cycloalkyl, heterocycle, cycloalkylalkyl, alkylcycloalkylalkyl, heterocycloalkyl or alkylheterocycloalkyl, which may optionally substituted with alkyl, OR⁸, COOR⁸, NR⁸R⁹, or NCOR⁸ moieties;
R⁸ and R⁹ are independently H or a lower alkyl group; and
B is O or S.

14. The method of claim 13 wherein the compound is selected from among (Z)-methyl 3-(((4-((2-(ethyl(methyl)amino)-2-oxoethyl)(methyl)amino)phenyl)amino)(phenyl)methylene)-2-oxo-2,3-dihydro-1H-pyrrolo[3,2-b]pyridine-6-carboxylate; (Z)-methyl 3-(((4-((3-(dimethylamino)-3-oxopropyl)(methyl)amino)phenyl)amino)(phenyl)methylene)-2-oxo-2,3-dihydro-1H-pyrrolo[3,2-b]pyridine-6-carboxylate; (Z)-methyl 3-(((4-(2-(1,1-dioxidothiomorpholino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(2-(dimethylamino)-N-methylacetamido)phenyl)amino)(phenyl)methylene)-2-oxo-2,3-dihydro-1H-pyrrolo[3,2-b]pyridine-6-carboxylate; (Z)-methyl 3-(((4-(N-methyl-2-(4-methylpiperazin-1-yl)acetamido)phenyl)amino)(phenyl)methylene)-2-oxo-2,3-dihydro-1H-pyrrolo[3,2-b]pyridine-6-carboxylate; and (Z)-methyl 3-(((4-(methyl(2-(4-methylpiperazin-1-yl)ethyl)amino)phenyl)amino)(phenyl)methylene)-2-oxo-2,3-dihydro-1H-pyrrolo[3,2-b]pyridine-6-carboxylate.
